(19) United States
(12) Patent Application Publication
(10) Pub. No.: US 2012/0310103 A1
(43) Pub. Date: Dec. 6, 2012

(54) HEART MONITOR WITH USER INPUT

(75) Inventors: Torsten MUSIOL, Ratingen (DE); David William LEWIS, Atlanta, GA (US); Marcos Ulises TONG ALVAREZ, Espoo (FI); Ole REINARTZ, Neuss (DE); Remberto MARTINEZ GONZALEZ, Pinsio (FI); Vreddhi BHAT, Bangalore (IN)

(73) Assignee: NOKIA SIEMENS NETWORKS OY, Espoo (FI)

(21) Appl. No.: 13/151,398

(22) Filed: Jun. 2, 2011

(51) Int. Cl. A61B 5/0404

(52) U.S. Cl. 600/523

(57) ABSTRACT

The invention provides a simple low-cost ECG monitoring device connected to a server (typically cloud based) via a mobile network with a mobile phone acting as a gateway. The invention enables the user to use the mobile communication device to provide additional data relating to how they are feeling. This user data is stored together with a timestamp so that it can be directly compared with the corresponding ECG data.

The present invention is related to the collection and use of electrocardiography (ECG) data.

Cardiovascular disease (CVD) is the number one cause of death globally. By 2030, 40.5% of the US population is projected to have some form of CVD. Between 2010 and 2030, real total direct medical costs of CVD are projected to triple, from $273 billion to $818 billion. Real indirect costs (due to lost productivity) for all CVD are estimated to increase from $172 billion in 2010 to $276 billion in 2030, an increase of 61%.

CVD incidents are usually associated with cardiac arrhythmias. On the other hand, issues related to cardiac arrhythmia risk do not only apply to persons with known cardiac disease or after a heart attack, but there are many other risk factors for cardiovascular diseases and sudden cardiac death.

The number of out-of-hospital sudden cardiac arrests (SCA) is significant. According to a study made in UK, 74% of all fatal events occurred outside hospital. Fewer than eight percent of people who suffer cardiac arrest outside the hospital survive.

In case of suspected heart issues, patients usually need to remain in hospital for ECG monitoring, or have to use an expensive home monitoring unit (event recorder).

As is well known in the art, electrocardiograph (ECG) techniques monitor the electrical activity of the heart. A typical ECG tracing of the cardiac cycle (heartbeat) consists of a P wave, a QRS complex and a T wave.

For ECG interpretation, the P, QRS and T waves are analyzed in terms of amplitude, duration, intervals between peaks and valleys and changes over time. Very often, rhythm events do not occur continuously, but require long observation time (perhaps one or more days).

A complete ECG analysis requires measurement of 12 voltages between different locations on the human body (12-lead ECG). In one embodiment of the invention, in order to meet the target of low cost and easy usability, a known single-lead ECG sensor is used. Single-lead ECG sensors detect many, but not all, heart anomalies. Clearly, any suitable ECG sensor, such as known 3-lead, 5-lead and 12-lead sensors could be used in embodiments of the present invention.

In addition to electrical measurement, acceleration measurement is performed in order to detect physical movement of the patient. This information is used to adjust thresholds for feedback notifications dynamically.

Doctor resources today are stretched with unnecessary visits from patients. It is also clear that an aging population is placing further burden on health care resources. On the other hand, there is a growing trend with consumers wanting to independently control and manage their own healthcare. No market solution is currently available to provide mobility to patients with real time feedback such as warning of critical events or issues.

Existing ECG monitoring systems also do not enable patients to provide additional inputs based on their own perceptions of their state of health.

The present invention seeks to address at least some of the problems outlined above.

The present invention provides a mobile communication device comprising: a first input configured to receive electrocardiography data of a user of the mobile communication device; a second input configured to receive additional data from said user; and a first output configured to provide said electrocardiography data and said additional data to a remote server via a mobile communications link.

An emergency input key may be provided to enable the use the additional data to indicate an emergency event. Alternatively, or in addition, a user interface may be provided to enable the user to provide said additional data. Such additional data may includes one or more pre-defined comments (such as “I feel dizzy”, “I feel faint”, “I feel murmur” and “I feel good”). Alternatively, or in addition, the additional data may include a free text option.

The present invention also provides a method comprising: receiving electrocardiography data of a user at a first input of a mobile communication device; receiving additional data from said user at a second input of the mobile communication device, wherein said input is provided directly by the user; and providing said electrocardiography data and said additional data to a (remote) server via a mobile communications link.

The additional data may include an indication of an emergency event.

The additional data may be provided using a user interface of the mobile communication device.

The present invention further provides an application (typically for a mobile communication device) comprising: a first input configured to receive electrocardiography data of a user of the mobile communication device; a second input configured to receive additional data from said user; and a first output configured to provide said electrocardiography data and said additional data to a remote server via a mobile communications link. The application may include a user interface to enable the user to provide said additional data.

The present invention yet further provides a computer program comprising: code (or some other means) for receiving electrocardiography data of a user at a first input of a mobile communication device; code (or some other means) for receiving additional data from said user at a second input of the mobile communication device, wherein said input is typically provided directly by the user; and code (or some other means) for providing said electrocardiography data and said additional data to a (remote) server via a mobile communications link. The computer program may be a computer program product comprising a computer-readable medium bearing computer program code embodied therein for use with a computer.

In many forms of the invention, the electrocardiography data and the additional data are stored with timestamp data such that the time of generation of the electrocardiography data can be compared with the time of generation of the additional data. The electrocardiography data and the additional data are typically stored in the same database and made available to doctor(s) together.

Exemplary embodiments of the invention are described below, by way of example only, with reference to the following numbered drawings.

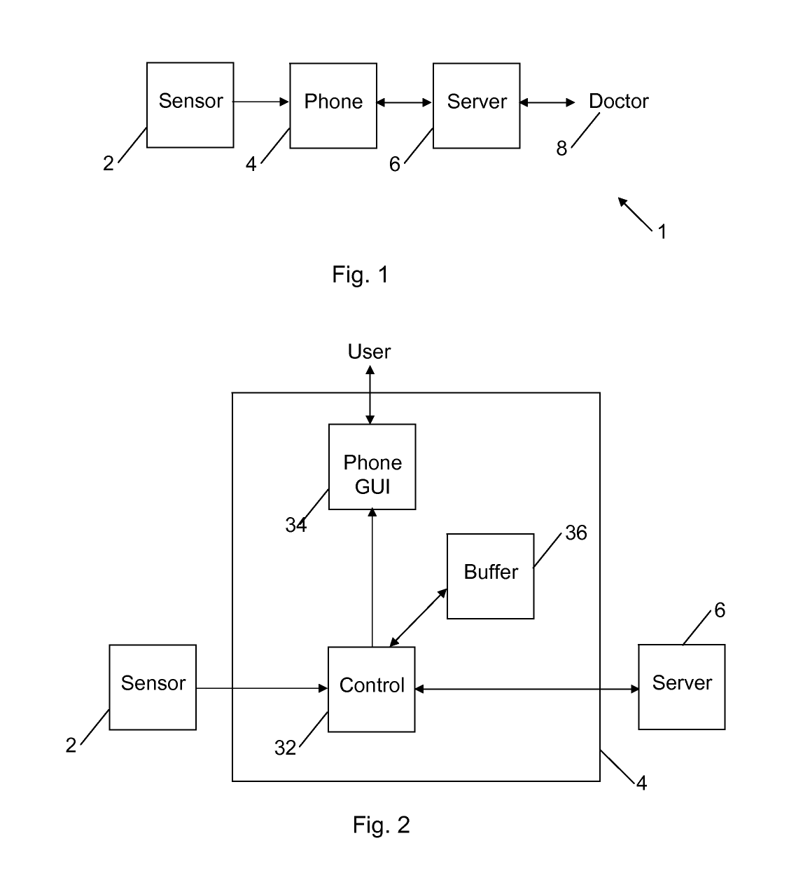

FIG. 1 is a block diagram of a system in accordance with an aspect of the present invention;

FIG. 2 is a block diagram showing further details of the system of FIG. 1;

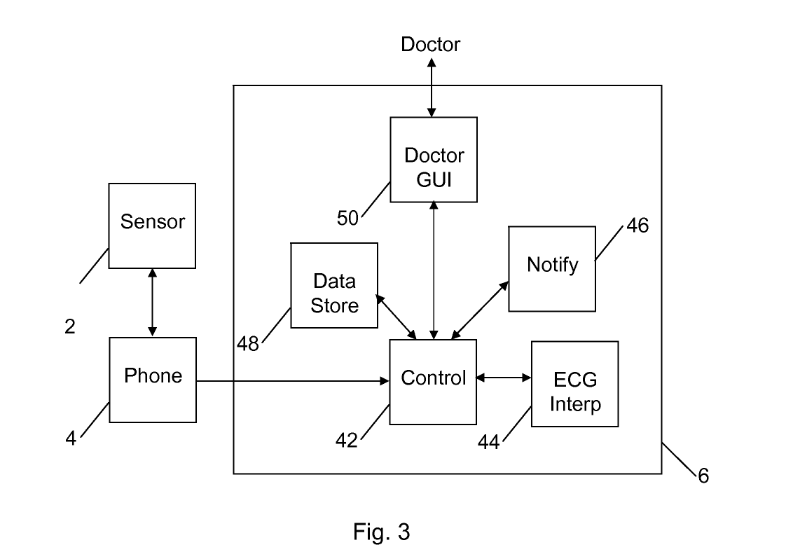

FIG. 3 is a block diagram showing further details of the system of FIG. 1; and

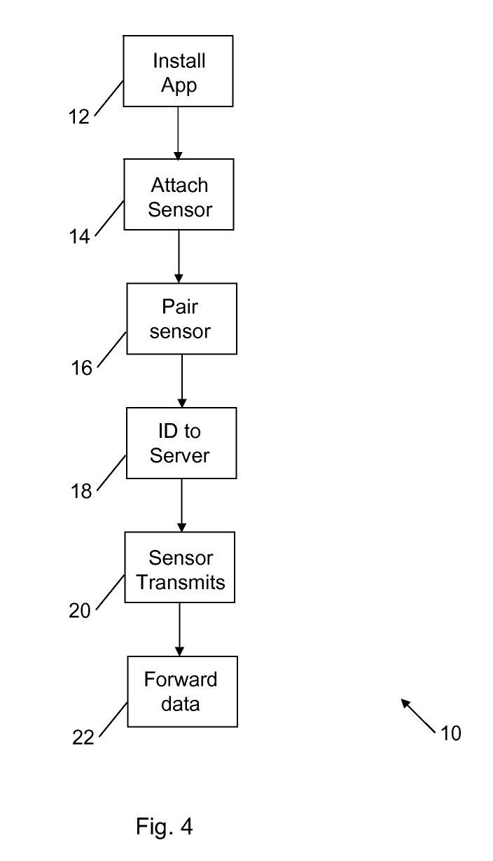

FIG. 4 is a flow chart showing an exemplary use of the system of FIG. 1; and

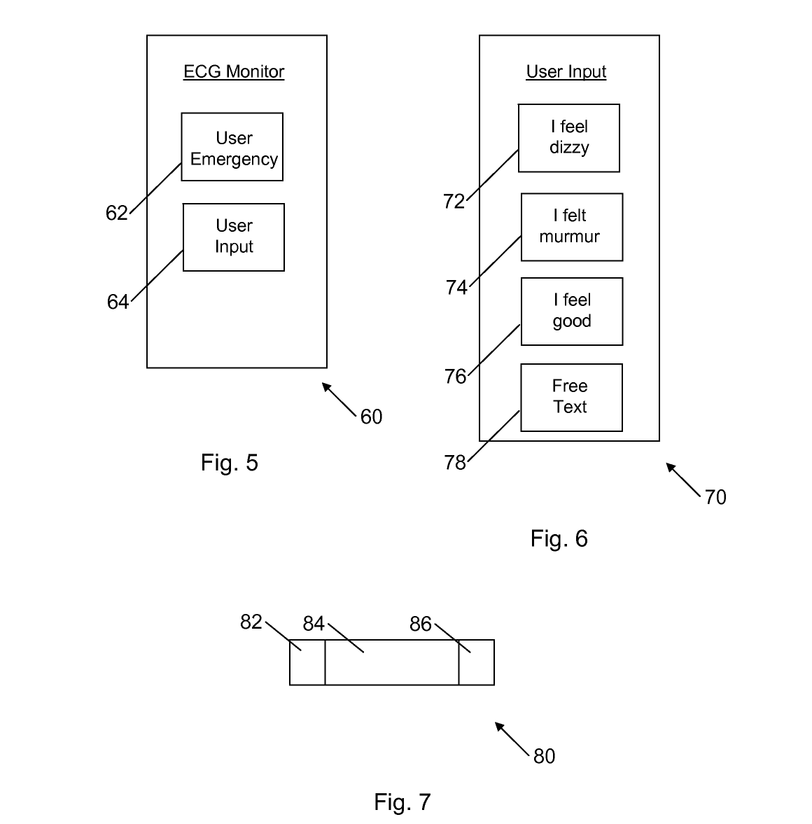

FIG. 5 shows a user interface in accordance with an aspect of the present invention;

FIG. 6 shows a user interface in accordance with an aspect of the present invention; and

FIG. 7 shows a data packet in accordance with an aspect of the present invention.

FIG. 1 is a block diagram of a system, indicated generally by the reference numeral 1, in accordance with an aspect of the present invention.

The system 1 comprises one or more sensors 2, a mobile communication device 4, and a server 6 and may additionally include a doctor 8. The sensor(s) 2 provide data to the mobile communication device 4. The device 4 is in two-way communication with the server 6 and so is able to upload data received from the sensor 2 to the server 6. The doctor 8 (when present in the system 1) is in two-way communication with the server 6 and can therefore access data uploaded to the server 6 by the mobile communication device 4.

The sensor 2 is an electrocardiography (ECG) sensor; however, the ECG sensor 2 may take many different forms. Indeed, one of the advantages of the present invention is that the system is sufficiently flexible to allow any suitable sensor to be used. Exemplary sensors 2 may, however, be chosen to meet at least some of the following criteria:

Single-lead ECG measurement

Acceleration measurement

Lead-off detection (whether the sensor is properly attached)

Battery supervision

Wireless connectivity to the mobile communication device 4

Low cost

Easy to handle by the user

Long battery lifetime (several days continuous operation)

Due to long-term usage, a sealed package is ideal.

FIG. 2 is a further block diagram showing the sensor 2, mobile communication device 4 and server 6 of the system 1 and additionally showing further details of the mobile communication device 4. As shown in FIG. 2, the mobile communication device includes a controller 32 that receives data from the sensor 2 and is in two-way communication with the server 6. The device 4 also includes a graphical user interface (GUI) 34 and a buffer 36 that are each in two-way communication with the controller 32. The GUI 34 enables the user (i.e. the subject of the monitoring by the sensor 2) to interact with the mobile communication device 4.

The device 4 typically supports at least some of the following functionality: pairing with the sensor 2; reception of ECG, impedance and acceleration measurement data from the sensor 2; display of ECG measurement data in a sliding window of the GUI 34; buffering (using the buffer 36) of measurement data with respect to the configurable data upload frequency; uploading of measurement data to the server 6; notifying the user if network connectivity is interrupted (WAN supervision), sensor connectivity is interrupted, in particular if the phone is not in proximity of the patient (PAN supervision), if the sensor device is not properly attached (lead-off detection) or if the sensor battery needs to be replaced or recharged; and notification to the user of ECG interpretation results (via the GUI 34). Many of these features are discussed further below.

FIG. 3 is a further block diagram showing the sensor 2, the mobile communication device 4 and the server 6 of the system 1 and additionally showing further details of the server 6. As shown in FIG. 3, the server 6 includes a controller 42, an ECG interpreter 44, a notification engine 46, a data store 48 and a graphical user interface (GUI) 50 for the doctor. The controller 42 is in two-way communication with the mobile communication device 4, the ECG interpreter 44, the notification engine 46, the data store 48 and the GUI 50. The doctor 8 interfaces with the server 6 via a two-way connection with the GUI 50.

In use, the mobile communication device 4 receives data from the sensor 2 and forwards that data (in a format discussed further below) to the controller 42 of the server 6. The controller 42 communicates with the data store 48 to store the data.

Data is sent from the controller 42 to the ECG interpreter 44 for analysis and results are returned to the controller 42. The results obtained from the ECG interpreter 42 are typically also stored in the data store 48. The doctor 8 uses the GUI 50 to access the data stored in the data store 48. Thus, the doctor can gain access to both the raw data received at the server 6 from the mobile communication device 2 and the results obtained from the ECG interpreter 44.

In some cases, the controller 42 may determine that a user (e.g. the subject of the monitoring by the sensor 2 of the doctor 8) should be informed of an event (such as an arrhythmia detected by the ECG interpreter 44 or a problem noted by the doctor 8). In this case, the controller 42 communications with a notification engine 46 and the engine provides a message for sending to the user (typically to the mobile communication device 4).

At least some of the elements of the server 6 may be provided remotely from the server. For example, the ECG interpreter 44 may be provided by a third party, with the server 6 sending data to the ECG interpreter and the ECG interpreter returning results to the controller 42 of the server 6. Similarly, data storage, such as the data store 48 may be provided remotely.

The server application software correlates the measured ECG data with the acceleration data and identifies heart rhythm anomalies (arrhythmia). This function is known as ECG interpretation.

FIG. 4 is a flow chart, indicated generally by the reference numeral 10, showing an exemplary use of the system 1.

The algorithm 10 starts at step 12, where the patient installs the relevant application on his mobile communication device 4. Next, at step 14, the patient attaches the sensor 2 to his chest.

The newly-attached sensor 2 needs to be paired with the mobile communication device 4 that the patient will use to upload data to the server 6. This is done in step 16 and need be done only once. Subsequently, the connection between the sensor 2 and the mobile communication device 4 is established automatically.

Next, at step 18, the patient logs into the server 6 using the application installed on his mobile communication device in step 12 above using credentials (username, password) as provided, for example, by the doctor 8.

At this stage, the sensor 2 is paired with the mobile communication device 4. Accordingly, at step 20, ECG measurement data is wirelessly transmitted from the sensor 2 to the mobile communication device 4. Next, at step 22, the data received at the mobile communication device 4 from the sensor 2 is transmitted to the server 6. Steps 20 and 22 are repeated for the duration of the measurement period.

Depending on the risk position of the patient and the actual medical need, the following sub-use cases (applications) are supported: very-long-term ECG (non-real-time); fast response (near real-time); and on demand (real-time).

On-demand ECG sends data continuously from the device 4 to the server 6 and supports remote diagnosis without a visit to the doctor.

The fast response ECG is further enhanced by a high upload frequency (e.g. once per minute). Continuous automatic ECG interpretation allows for fast response in case of an apparently dangerous situation for the user/patient. This application requires more resources, in particular battery power from the mobile phone and a consistent network connection. During phases of network unavailability, the data will be stored on the mobile phone.

Very-long-term ECG is a conventional long-term ECG application, enhanced by virtually infinite observation time and characterized by significantly lower costs. For optimum usage of mobile phone resources, data is uploaded with low frequency (e.g. once per day). This use case is applicable to the family doctor as well as the clinician.

FIG. 5 shows a user interface, indicated generally by the reference numeral 60, in accordance with an aspect of the present invention.

The user interface 60 includes a “user emergency” button enabling the user of the mobile communication device 4 (i.e. the person being monitored by the sensor) to indicate an emergency event. The provision of a single button enables an emergency event (such as a heart attack) to be indicated by the user with the minimum of effort, delay and concentration. The indication of an emergency event may result in an alert being provided to a doctor and/or to a third party (such as an emergency contact or a paramedic). The emergency alert may provide location data relating to the user to the doctor, paramedic and/or emergency contact.

The user interface 60 also includes a “user input” button 64. The user input button is used to allow the user to provide information outside of emergency situations (when the user has more time to interact with the user interface 60). Pressing the user input button 64 takes the user to the user interface 70 shown in FIG. 6.

The user interface 70 includes an “I feel dizzy” button 72, an “I felt murmur” button 74, an “I feel good” button 76 and a “Free text” button 78. Pressing any of the buttons 72, 74 or 76 results in the corresponding data being input to the data stream sent to the server 6. Pressing the button 78 enables the user to enter any text that they wish.

Of course, more or different options to those shown in FIG. 6 could readily be provided.

The user interfaces 60 and 70 are provided by way of example only. The skilled person will be aware of many alternative mechanisms that would also the user to provide such data to the server 6.

FIG. 7 shows a data structure, indicated generally by the reference numeral 80, in accordance with an aspect of the present invention. The data structure 80 comprises a timestamp 82, a first data portion 84 and a second data portion 86. The first data portion 84 includes data provided by the sensor 2. The second data portion 86 includes user event data as input using the user interface 60 or the user interface 70. The second data portion 86 may be empty if the user has not provided any input.

The data structure 80 enables user input, such as “I feel dizzy” to be matched with the ECG data being generated at the time. This enables a doctor to review the data and to make a more complete analysis that might have been possible based on the ECG data alone.

The system 1 provides a solution for both individuals and doctors, built upon low-cost ECG monitoring devices that are connected to the network via the mobile phone of the user and a Cloud based server architecture. Users have full mobility and heart rhythms are continually monitored with near “real time” feedback from an analytical engine being provided, if required. The solution supports continual recording, storage and processing of information for doctors. It automatically alerts the patient, first responders, doctors or caregivers of any major rhythm event.

Two exemplary use cases of the system 1 are described below.

The first use case is intended largely for use by doctors. ECG data is recorded by the system and the doctor can access the recorded data using the GUI 50 described above. The doctor can also access user generated data provided using the user interfaces 60 and 70. In addition, the ECG interpreter 44 can alert the doctor in the event that potential problems (such as arrhythmia events) are detected.

The second use case is intended largely for use by individuals. The system 1 supports self-monitoring by the user (preventive care). This is facilitated by the ECG interpreter 44 running autonomously on the server 6. The server 6 notifies the user instantly if anomalies exceed a certain threshold and the user should visit the doctor. In case of danger to life the system 1 may also alert the emergency services and other caregivers (e.g. relatives or neighbors) nominated by the user. The user may provide his doctor access to his data.

As described above, the invention provides a simple low-cost ECG monitoring device connected to a server (typically cloud based) via a mobile network with a mobile phone acting as a gateway.

The remote software can analyse the data. Raw data, and analysed results, are stored in bulk remote from the sensor (e.g. in the cloud). The doctor has access to this data without requiring the patient to be present (and has access to data generated after the patient's last visit to the doctor).

The basic system architecture involves a sensor device, a mobile phone and a server. The sensor device is typically an “off-the-shelf” device, such as a digital plaster. The sensor communicates with a paired mobile phone in a very simple and well-established manner. The mobile phone has the relevant software installed. Data is received from the sensor and sent to the server; data buffering may be required (e.g. if connection to the server is lost). A data display (to the user) may be provided, but this is not essential. User notification (e.g. of alerts) may be provided. The server may require secure login and may have the bulk data storage and the main data processing capability of the system. The server typically provides the ECG interpretation, performs data plotting and issues alerts (if such a feature is provided by the system). The server may need to interface with multiple users (e.g. the patient, doctors, paramedics, relatives, emergency contacts).

Advantages of the invention include the following. Each part of the system can be optimized. The sensor can be as simple as possible (just provides data—no need for data processing); thus the sensor can be cheap and battery usage minimized. The communication system is optimized by allowing mobile phone operators to do all the work (e.g. redundancy by providing multiple communication methods). The storage in the cloud is cheap. The centralized software (rather than providing software to the phones) is cheaper, simpler and easier to update. The system enables long observations times that provide a clear medical advantage. The system is universal and scalable. The system is also flexible, allowing new applications/modified applications to be provided (e.g. by others) as required. Doctors have access to bulk data stored at the server regardless of whether the patient is present. Paramedics can also potentially access bulk data (e.g. via a similar GUI to that available to a doctor).

The main benefit for the individual is higher quality of life, a patient who is post operative or has post event condition (e.g. heart attack) is able to experience a quick, easy and safe reintegration into their home environment. A patient with the concern of a heart related disease can continue their private and professional routine as a result of being able to monitor their situation. Since the patient can stay at home, the so-called “white coat syndrome” is eliminated and occupational rehabilitation costs will be reduced.

There are benefits for the doctor as well. ECG monitoring costs can be significantly reduced through low-cost devices and simpler handling. Longer observation time supports a high quality of diagnosis. Cloud based computing with secure web access keeps infrastructure costs low.

The embodiments of the invention described above are illustrative rather than restrictive. It will be apparent to those skilled in the art that the above devices and methods may incorporate a number of modifications without departing from the general scope of the invention. It is intended to include all such modifications within the scope of the invention insofar as they fall within the scope of the appended claims.

1. A mobile communication device, comprising:
a first input configured to receive electrocardiography data of a user of the mobile communication device;
a second input configured to receive additional data communicated from said user, said additional data designated to be matched with the electrocardiography data; and
a first output configured to provide said electrocardiography data and said additional data to a remote server via a mobile communications link.

2. A mobile communication device as claimed in claim 1, further comprising a user interface configured to enable the user to provide said additional data.

3. A mobile communication device as claimed in claim 2, wherein the user interface is further configured to enable to provide the additional data including one or more pre-defined comments.

4. A mobile communication device as claimed in claim 1, further comprising:
an emergency input key configured to enable the user to use the additional data to indicate an emergency event.

5. A mobile communication device as claimed in claim 1, further comprising:
a memory configured to store the electrocardiography data and the additional data with timestamp data, wherein the timestamp data is configured to permit the time of generation of the electrocardiography data to be compared with the time of generation of the additional data.

6. A method, comprising:
receiving electrocardiography data of a user at a first input of a mobile communication device;
receiving additional data communicated from said user at a second input of the mobile communication device, said additional data designated to be matched with the electrocardiography data; and
providing said electrocardiography data and said additional data to a server via a mobile communications link.

7. A method as claimed in claim 6, wherein receiving said additional data includes receiving an indication of an emergency event.

8. A method as claimed in claim 6, wherein receiving said additional data comprises receiving said additional data provided using a user interface of the mobile communication device.

9. A method as claimed in claim 6, further comprising:
storing the electrocardiography data and the additional data with timestamp data such that the time of generation of the electrocardiography data; and
comparing at least one of the electrocardiography data or the additional data with the time of generation of the additional data.

10. An application, comprising:
a first input configured to receive electrocardiography data of a user of the application at a mobile communication device;
a second input configured to receive additional data communicated from said user, said additional data designated to be matched with the electrocardiography data; and
a first output configured to provide said electrocardiography data and said additional data to a remote server via a mobile communications link, wherein the application is implemented at least in part in hardware.

11. An application as claimed in claim 10, further comprising a user interface configured to enable the user to provide said additional data.

12. A non-transitory computer program product comprising computer readable executable code, when run on a processor, controls said processor to perform a method comprising:
receiving electrocardiography data of a user at a first input of a mobile communication device;
receiving additional data communicated from said user at a second input of the mobile communication device, said additional data designated to be matched with the electrocardiography data; and
providing said electrocardiography data and said additional data to a server via a mobile communications link.

13. A mobile communication device as claimed in claim 1, wherein said first output is configured to provide said electrocardiography data and said additional data to the remote server in a same data stream.
